(19) United States
(12) Patent Application Publication
(10) Pub. No.: US 2010/0015111 A1
(43) Pub. Date: Jan. 21, 2010

(54) Methods And Kits For The Treatment Inhibition, And Maintenance Of Gastrointestinal Disorders

(76) Inventors: Simon Henry Magowan, Loveland, OH (US); Linda Mary Law, Cincinnati, OH (US); Karla Ann Hess Horstman, Liberty Township, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/491,522

(22) Filed: Jun. 25, 2009

Related U.S. Application Data
(60) Provisional application No. 61/076,207, filed on Jun. 27, 2008.

(51) Int. Cl. A61K 35/74; A61K 31/60; A61P 1/00

(52) U.S. Cl. 424/93.45; 514/159; 424/93.4

(57) ABSTRACT

The present invention comprises methods and kits that are useful treatment, inhibition and or maintenance of gastrointestinal disorders including inflammatory bowel disorder. The methods comprise: commencing treatment by orally administering a first dose of a pharmaceutical composition comprising from about 1 mg to about 8000 mg of an active agent and continuing said treatment, inhibition and or maintenance by orally administering an additional dose on a less than daily schedule of an active agent., either alone or in combination with a probiotic or optionally an antibiotic or optionally a prebiotic.

### CROSS REFERENCE TO RELATED APPLICATIONS

This application claims the benefit of U.S. Provisional Application No. 61/076,207, filed on Jun. 27, 2008.

### FIELD OF THE INVENTION

The present invention is directed to methods and kits for the treatment, inhibition and or maintenance of gastrointestinal disorders including inflammatory bowel disorder. The methods are directed to treating, inhibiting, and/or maintaining remission of a gastrointestinal disorder comprising: commencing treatment by orally administering a first dose of a pharmaceutical composition comprising from about 1 mg to about 8000 mg of an active agent and continuing said treatment, inhibition and or maintenance by orally administering an additional dose on a less than daily schedule of an active agent, either alone or in combination with a probiotic or optionally an antibiotic or optionally a prebiotic.

### BACKGROUND OF THE INVENTION

Health care product users experiencing functional digestive conditions, including irritable bowel syndrome and/or inflammatory bowel disorders, may have a wide range of symptoms. These symptoms may occur often, but the precise frequency and severity may be unpredictable, resulting in stress, a feeling of lack of control, all of which may significantly impact the user's quality of life and compliance with the prescription written by the health care professional. In addition, these users are often disappointed with the medical care received, as certain functional disorders are by nature diagnoses of exclusion and these users are often told that nothing is wrong with them.

The large bowel in humans and to a lesser extent the small bowel, contain large concentrations of various enteric bacteria. Generally, patients will have no pain, cramping, diarrhea, or constipation if the bacterial contents are not infected with pathogenic strains which may colonize the bowel and remain there for prolonged periods of time. Acute infections and some chronic infections of the bowel flora can however cause inflammatory changes in the lining and in the deeper layers of the colonic wall. When inflammation is visible in the deeper layer of bowel wall and is associated with irritable bowel symptoms, the condition is called inflammatory bowel disorder and includes such diseases as inflammatory irritable bowel syndrome and microscopic colitis. These diseases are conditions recognized and known to arise from an inflammatory condition of the colon. In particular, inflammatory irritable bowel syndrome and microscopic colitis are known to be caused by a precipitating infection by a pathogenic organism or organisms. The degree of inflammation in inflammatory irritable bowel syndrome and microscopic colitis is much less than inflammatory bowel diseases such as ulcerative colitis and/or Crohn's disease.

Normally, there are no visible colonoscopic abnormalities in inflammatory bowel disorders that include inflammatory irritable bowel syndrome and/or microscopic colitis. Instead, inflammatory bowel disorders are characterized by inflammation on histology. Inflammatory irritable bowel syndrome and microscopic colitis show evidence of inflammation on histology obtained by colonoscopy and the histology shows an increase in inflammatory cells. Inflammatory irritable bowel syndrome and microscopic colitis can be referred to as specific bowel disorders because there are specific diagnostic criteria such as inflammation in the wall of the colon that can help diagnose it. Stool samples can be collected and the stool tests are positive for inflammatory markers of inflammation, and the patient complains of symptoms referable to the colon, such as urgency, diarrhea, flatulence, cramping, the diagnosis of inflammatory irritable bowel syndrome or microscopic colitis can also be made. Collectively, between 5% and 25% of the western population in different age groups may suffer from these disorders which have also been termed spastic colon, unstable colonic neurosis, spastic colitis, or mucous colitis. In a classic case there is a triad of symptoms including low abdominal pain relieved by defecation, alternating constipation/diarrhea and the passage of small caliber stools. In some patients there may be accompanying watery diarrhea with or without pain. Distension, flatulence, wind and at times nausea and headaches may also be accompanying systemic symptoms. Some patients also experience at times diarrhea alternating with constipation.

To date, there is no therapy that has addressed the inflammatory pathogenesis of inflammatory irritable bowel syndrome. Conventional treatments for Irritable Bowel Syndrome (IBS) have been unsatisfactory as exemplified by the large number of therapies that have from time to time been recommended or studied. These have included psychotherapy, dietary regimens, anti-spasmodic agents, anti-cholinergics, antidepressants, bulking agents, various receptor antagonists, carminatives, opiates, and tranquillizers, all without substantial success. Indeed, there is no evidence that a cure is possible. Yet IBS is one of the most common of all the gastrointestinal illnesses and though not life threatening causes great distress especially to those severely affected, and may bring a feeling of frustration and helplessness, being generally life long. In particular, diarrhea-predominant IBS can cause incontinence in some patients and, for example, the inability of being sure that one can reach ones employment causing some to drive from rest room to rest room on their way to work. In some patients urgency is so severe that they can only hold their motions for a few seconds.

Pharmaceutical compositions are widely used and may vary from products chosen by a user without the assistance of a health care professional or chosen as the result of a recommendation or prescription from a health care professional. Patient compliance with prescriptions from the health care professional continues to be an on going problem in the health care industry. It is believed that patients find that the need to take multiple doses multiple times a day for an extended period of time results in a burden to the user and therefore the user tends to not take the medicine as prescribed and/or miss doses, and/or stop taking the medicine altogether. It is believed that decreasing the amount of doses and frequency of the doses that a patient needs to administer to provide health benefits will assist in patient compliance with recommendations and prescription given by the health care professional and provide meaningful quality of life improvement as their symptoms are treated and inhibited.

The treatment, inhibition and or maintenance of gastrointestinal disorders including inflammatory bowel disorder may be provided per the various embodiments described herein by decreasing the amount of doses and/or frequency of doses of a pharmaceutical composition that a patient needs to administer to provide health benefits for example by administering a first does of a pharmaceutical composition comprising an effective amount of aminosalicylate, sulfasalazine, 5-aminosalicylic, 4-aminosalicylic acid, benzalazine, dihydrochloride salt, olsalazine, balsalazide, bismuth subsalicylate, and mixtures thereof and then following this dose with an additional dose on a less than daily schedule. Additionally, a therapeutic manipulation of the colonic flora may decrease colonic inflammation and ameliorate symptoms.

### SUMMARY OF THE INVENTION

One embodiment is directed to methods and kits for the treatment, inhibition and or maintenance of gastrointestinal disorders including inflammatory bowel disorder. In one embodiment, the invention is directed to a method for treating, inhibiting, and/or maintaining remission of a gastrointestinal disorder comprising: commencing treatment by orally administering a first dose of a pharmaceutical composition comprising from about 1 mg to about 8000 mg of an active agent and continuing said treatment, inhibition and or maintenance by orally administering an additional dose on a less than daily schedule of an active agent.

In yet another embodiment, the invention is directed to a method for treating, inhibiting, and/or maintaining remission of a gastrointestinal disorder comprising: commencing treatment by orally administering a pharmaceutical composition comprising an active agent on an every other day schedule; wherein said composition comprising from about 1 mg to about 8000 mg of said active agent.

In yet another embodiment, the invention is directed to a kit for use in a regimen for treating, inhibiting, and/or maintaining remission of a gastrointestinal disorder, said regimen comprising orally administering a first dose of a pharmaceutical composition comprising from about 1 mg to about 8000 mg of an active agent and continuing said treatment, inhibition and or maintaining remission by orally administering an additional dose on a less than daily schedule of an active agent, said kit comprising:
a. one dose of an active agent; and
b. from about one to about forty-five additional doses of an active agent.

In yet another embodiment, the invention is directed to a kit for use in a regimen for treating, inhibiting, and/or maintaining remission of a gastrointestinal disorder comprising: commencing treatment by orally administering a dose on an every other day schedule of a pharmaceutical composition comprising from about 1 mg to about 8000 mg of an active agent, and said kit further comprising from about one to about forty-five of an additional dose of an active agent given on an every other day schedule.

In yet another embodiment, the invention is directed to a gastro-intestinal composition comprising; from about 1 mg to about 8000 mg of an salicyclic derivative, at least about 10⁵ cfu of a probiotic; wherein said probiotic is selected from the group consisting of Lactobacillus spp., Bifidobacterium spp., or combinations thereof; and wherein said composition is orally administered on less than daily basis.

The kits and or methods may optionally comprise an antibiotic, a prebiotic and/or a probiotic.

### BRIEF DESCRIPTION OF THE DRAWINGS

FIG. 1a is a graph of the Body Weight of the Male Swiss-Webster mice treatment group over time;

FIG. 1b is a graph of the change of Body Weight of Male Swiss-Webster mice treatment group from day −3(g);

FIG. 2 is a graph of the percent survival of Male Swiss-Webster mice treatment group;

FIG. 3 is a graph of the colon length of Male Swiss-Webster mice treatment group;

FIG. 4 is a graph of the colon score of Male Swiss-Webster mice treatment group;

FIG. 5 is a graph of the colon inflammation score of Male Swiss-Webster mice treatment group;

FIG. 6 is a graph of the colon gland loss score of Male Swiss-Webster mice treatment group;

FIG. 7 is a graph of the colon erosion score of Male Swiss-Webster mice treatment group;

FIG. 8 is a graph of the histopathology sum of Male Swiss-Webster mice treatment group;

FIG. 9 is a graph of the edema of the of Male Swiss-Webster mice treatment group;

FIG. 10 is a graph of the mucosal thickness of the of Male Swiss-Webster mice treatment group; and

FIG. 11 is a graph of the hyperplasia score of Male Swiss-Webster mice treatment group.

### DETAILED DESCRIPTION OF THE INVENTION

The present invention comprises methods and kits that are useful for treatment, inhibition and or maintenance of gastrointestinal disorders including for example inflammatory bowel disorder.

As used herein, the term “administration”, “administering”, “or the like with respect to the user means that the user is administered, is directed to administer or, with reference specifically to “oral administration,” or “orally administering,” ingests or is directed to ingest, the composition. For example, the administration may be oral administration, parenteral administration, topical administration, buccal administration, rectal administration, or the like, or any combination thereof. As but one example, the active agent may be administered orally or rectally, while the probiotic may be administered orally. In an embodiment, all components are administered through oral administration.

As used herein, the term “inflammatory bowel disorder”, includes a disorder that is diagnosed by at least one of the following criteria 1) inflammation in the wall of the colon and/or 2) a stool sample that tests positive for inflammatory markers of inflammation in combination with a patient that complains of at least one symptom. The symptoms include but are not limited to low abdominal pain relieved by defecation, alternating constipation/diarrhea, passage of small caliber stools, cramping, diarrhea, constipation, urgency, flatulence, watery diarrhea with or without pain, distension, nausea and or incontinence. Inflammatory bowel disorder includes inflammatory irritable bowel syndrome, irritable bowel syndrome-diarrhea, irritable bowel syndrome-constipation, irritable bowel syndrome-mixed, irritable bowel syndrome-alternating, dyspepsia, microscopic colitis, lymphocytic colitis, collagenous colitis, indeterminant colitis, celiac disease, and combinations thereof.

As used herein, the term “inhibition” refers to the repression of symptoms of the active disease state and/or medical condition and/or gastrointestinal disorders.

As used herein, the term maintenance refers to the administration of the composition starting from about 90 days after the initial acute flare-up phase of the disease state and/or medical condition and/or gastrointestinal disorders, alternatively administration of the composition starting from about 100 days after the initial acute flare-up phase of the disease state and/or medical condition and/or gastrointestinal disorders, alternatively administration of the composition starting from about 120 days after the initial acute flare-up phase of the disease state and/or medical condition and/or gastrointestinal disorders.

As used herein, the term “less than daily dose” and/or “less than daily schedule” includes administration of the pharmaceutical composition every other day, once weekly, twice weekly, once every two weeks, once monthly, once every other month, and combinations thereof.

As used herein, “treating” refers to the amelioration and/or delay of at least one symptom of a medical condition and in particular embodiments does not necessarily encompass a cure for the medical condition.

Except where specific examples of actual measured values are presented, numerical values referred to herein should be considered to be qualified by the word “about”.

All weights, measurements and concentrations herein are measured at 25° C. on the composition in its entirety, unless otherwise specified.

These and other limitations of the compositions and methods of the present invention, as well as many of the optional ingredients suitable for use herein, are described in detail hereinafter.

All percentages, parts and ratios as used herein are by weight of the total composition, unless otherwise specified. All such weights as they pertain to listed ingredients are based on the active level and, therefore do not include solvents or by-products that may be included in commercially available materials, unless otherwise specified.

The composition methods, and/or kits of the present invention can comprise, consist of, or consist essentially of, the essential elements and limitations of the invention described herein, as well as any additional or optional ingredients, components, or limitations described herein or otherwise useful in compositions intended for use by a mammal, preferably human use.

### Methods of the Present Invention

The present invention is directed to methods and kits for treatment, inhibition and or maintenance of gastrointestinal disorders including inflammatory bowel disorder.

In one embodiment, the invention is directed to a method for treating, inhibiting, and/or maintaining remission of a gastrointestinal disorder selected from inflammatory bowel disease, Crohn's disease, ulcerative colitis, inflammatory bowel disorder, irritable bowel syndrome, irritable bowel syndrome-diarrhea, irritable bowel syndrome-constipation, irritable bowel syndrome-mixed, irritable bowel syndrome-alternating, dyspepsia, gastro-esophageal reflux, diverticulitis, diverticular disease, gastroparesis, microscopic colitis, lymphocytic colitis, collagenous colitis, indeterminant colitis, eosinophilic esophagitis, HIV-associated diarrhea, antibiotic-associated colitis, clostridium difficile-associated diarrhea, pseudo-membranous colitis, diarrhea associated with immunodeficiency disorders, small bowel overgrowth syndrome, celiac disease, Whipple's disease, CMV-associated colitis, Behcet's syndrome, and combinations thereof comprising: commencing treatment by orally administering a first dose of a pharmaceutical composition comprising from about 1 mg to about 8000 mg of an active agent and continuing said treatment, inhibition and or maintenance by orally administering an additional dose on a less than daily schedule of an active agent and optionally a probitoic and/or a prebiotic and/or an antibiotic.

In one embodiment, the invention is directed to a method for treating, inhibiting, and/or maintaining remission of a gastrointestinal disorder from inflammatory irritable bowel syndrome, irritable bowel syndrome-diarrhea, irritable bowel syndrome-constipation, irritable bowel syndrome-mixed, irritable bowel syndrome-alternating, dyspepsia, microscopic colitis, lymphocytic colitis, collagenous colitis, indeterminant colitis, celiac disease, and combinations thereof, the method comprising: commencing treatment by orally administering a first dose of a pharmaceutical composition comprising from about 1 mg to about 8000 mg of an active agent and continuing said treatment, inhibition and or maintenance by orally administering an additional dose on a less than daily schedule of an active agent, and optionally a probiotic, and/or optionally an antibiotic, and/or optionally a prebiotic.

In one embodiment, the invention is directed to a method for treating, inhibiting, and/or maintaining remission of a gastrointestinal disorder of a gastrointestinal disorder selected from inflammatory bowel disease, Crohn's disease, ulcerative colitis, inflammatory bowel disorder, irritable bowel syndrome, irritable bowel syndrome-diarrhea, irritable bowel syndrome-constipation, irritable bowel syndrome-mixed, irritable bowel syndrome-alternating, dyspepsia, gastro-esophageal reflux, diverticulitis, diverticular disease, gastroparesis, microscopic colitis, lymphocytic colitis, collagenous colitis, indeterminant colitis, eosinophilic esophagitis, HIV-associated diarrhea, antibiotic-associated colitis, clostridium difficile-associated diarrhea, pseudo-membranous colitis, diarrhea associated with immunodeficiency disorders, small bowel overgrowth syndrome, celiac disease, Whipple's disease, CMV-associated colitis, Behcet's syndrome, and combinations thereof comprising: comprising: commencing treatment by orally administering a dose on an every other day schedule of a pharmaceutical composition comprising from about 1 mg to about 8000 mg of an active agent.

Inflammatory bowel disorder may be associated with a variety of symptoms including, for example, in a classic case a triad of symptoms including low abdominal pain relieved by defecation, alternating constipation/diarrhea and the passage of small caliber stools. In some patients there may be accompanying watery diarrhea with or without pain. Distension, flatulence, wind and at times nausea and headaches may also be accompanying systemic symptoms. Some patients also experience at times diarrhea alternating with constipation. The methods herein encompass treatment of any of these variety of symptoms.

Additionally, inflammatory bowel disorders are characterized by inflammation on histology. Inflammatory irritable bowel syndrome and microscopic colitis show evidence of inflammation on histology obtained by colonoscopy and the histology shows an increase in inflammatory cells. Stool samples can also be collected and the stool tests are positive for inflammatory markers of inflammation, and the patient complains of symptoms referable to the colon, such as urgency, diarrhea, flatulence, cramping, the diagnosis of inflammatory irritable bowel syndrome or microscopic colitis can also be made.

Diverticular conditions may be associated with a variety of symptoms including, for example, abdominal pain, abdominal tenderness, nausea, vomiting, bloating, constipation, diarrhea, mucus in stool, feeling urge to evacuate but no bowel movement, painful straining with bowel movement, and pain or difficulty urinating and, as such, the methods herein encompass treatment of any of these variety of symptoms.

Ulcerative colitis (UC) is a condition that causes inflammation and sores in the form of ulcers, in the lining of the rectum and colon and, as such, the methods herein encompass treatment of any of these variety of symptoms.

UC is a type of inflammatory bowel disease (IBD). IBD is the general name for diseases that cause inflammation in the small intestine and colon. UC is oftentimes difficult to diagnose as it shares symptoms common to other intestinal disorders and to Crohn's disease, another type of IBD. Crohn's disease differs because it causes inflammation deeper within the intestinal wall and can occur in other parts of the digestive system including the small intestine, mouth, esophagus, and stomach and, as such, the methods herein encompass treatment of any of this variety of symptoms.

Pharmaceutical Composition

The pharmaceutical compositions of the present invention comprises from about 1 mg to about 8000 mg of an active agent for example a salicyclic derivative, and optionally at least about 10⁵ cfu of a probiotic; wherein said probiotic can be selected from the group consisting of Lactobacillus spp., Bifidobacterium spp., or combinations thereof. Additionally, the pharmaceutical composition can comprise a prebiotic and/or an antibiotic.

The pharmaceutical composition can be administered as a first dose of a pharmaceutical composition comprising from about 1 mg to about 8000 mg of an active agent and continuing said treatment, inhibition and or maintenance by orally administering an additional dose on a less than daily schedule of an active agent

Wherein the user is directed to administer the composition or component, such direction may be that which instructs and/or informs the user that use of the composition or component (as applicable) may and/or will provide one or more general health and/or general physiological benefits associated with the health care product. For example, such direction may be oral direction (e.g., through oral instruction from, for example, a physician, health professional, sales professional or organization, and/or radio or television media (e.g., advertisement) or written direction (e.g., through written direction from, for example, a physician or other health professional (e.g., scripts), sales professional or organization (e.g., through, for example, marketing brochures, pamphlets, or other instructive paraphernalia), written media (e.g., internet, electronic mail, or other computer-related media), and/or containing devices associated with the composition (e.g., a label present on a containing device containing the composition). See e.g., the kits described herein.

Active Agent

The methods of the invention comprise administration of at least one active agent. Nonlimiting examples of the active agent may include those selected from aminosalicylate, sulfasalazine, 5-aminosalicylic acid, 4-aminosalicylic acid, benzalazine, dihydrochloride salt, olsalazine, balsalazide, bismuth subsalicylate, and mixtures thereof.

In one embodiment herein, the active agent may be an aminosalicylate. As used herein, “aminosalicylate” refers to a class of compounds capable of releasing 5-amino-2-hydroxybenzoate or 5-amino-2-hydroxybenzoic acid as an active moiety in vivo. Non-limiting examples include mesalamine(5-amino-2-hydroxybenzoic acid), olsalazine(3,3′-dicarboxy-4,4′-dihydroxyazobenzene), balsalazide((E)-5-[[4-[[(2-carboxyethyl)amino]carbonyl]phenyl]azo]-2-hydroxybenzoic acid), and sulfasalazine(2-hydroxy-5-[[4-[(2-pyridinylamino)sulfonyl]phenyl]azo]-benzoic acid).

A composition comprising an aminosalicylate may have one or greater than one aminosalicylate in addition to other possible components. The active moiety is illustrated below:

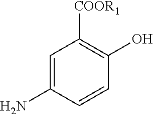

wherein R₁ can be hydrogen or a physiologically relevant counterion and the nitrogen can be further protonated and carry a positive charge along with a physiologically relevant counterion.

Although the examples provided describe the free acid or free amine forms, the term is not so limited and should be interpreted to include the free acid forms, the free amine forms, and any salts thereof. For example, the term “mesalamine” covers the free acid, the free amine, and any salts of mesalamine. The term “mesalamine” is commonly used interchangeably in the art with “mesalazine”, “5-ASA” or “5-aminosalicylic acid”.

In one embodiment herein, the active agent is formulated for release in the small intestines and/or the large intestine, such as for example at the distal portion of the small intestine and in the large intestine. Various illustrative commercial products are suitable for such release including, for example, ASACOL, PENTASA, and LIALDA. Various delayed or sustained delivery technologies are well known for this purpose and need not be described herein.

In accordance with the methods herein, as an example, from about 1 mg to about 8000 mg of the active agent may be administered, or from about 50 mg to about 7000 mg, or from about 100 mg to about 6000 mg, or from about 200 mg to about 4800 mg, or from about 1000 mg to about 4800 mg, or from about 1500 to about 4800 mg or from about 2400 mg to about 4800 mg. The active agent can be administered as a first dose and then followed up with additional dose. The first dose and/or additional dose can be administered on a less than daily schedule. In one embodiment herein, the active agent is administered as a first does and followed up with additional doses on a less than daily schedule wherein the less than daily schedule is every other day. In another embodiment the active agent is administered on an every other day schedule. When the active agent is administered as a first dose the first dose comprises from about 1 mg to about 8000 mg of the active agent may be administered, or from about 50 mg to about 7000 mg, or from about 100 mg to about 6000 mg, or from about 200 mg to about 4800 mg, or from about 1000 mg to about 4800 mg, or from about 1500 to about 4800 mg or from about 2400 mg to about 4800 mg. When the active agent is administered as an additional dose the additional dose comprises from about from about 200 mg to about 4800 mg, or from about 400 mg to about 4800 mg, or from about 500 mg to about 4800 mg, or from about 1500 to about 4800 mg or from about 2400 mg to about 4800 mg of said active ingredient.

In certain embodiments herein, the active agent is administered prior to administration of a probiotic. In this embodiment, the active agent is administered for a definite period of time (for illustration, three times day on an every other day schedule for a ten week period of time); upon conclusion of this illustrative ten week period of time, administration of a probiotic commences and continues for a definite period of time or, optionally, indefinitely.

In other embodiments herein, the active agent is administered contemporaneously with administration of a probiotic. As used herein, “contemporaneously” means that, for any given day of administration or less than daily dose schedule, the active agent and the probiotic are both administered on that day (whether at the same time, or at different times during that day).

In other embodiments herein, the active agent is administered contemporaneously with administration of a probiotic and/or prebiotic. As used herein, “contemporaneously” means that, for any given day of administration or less than daily dose schedule, the active agent and the probiotic and/or prebiotic are all administered on that day (whether at the same time, or at different times during that day).

In other embodiments herein, the active agent is administered prior to administration of a probiotic and is also administered contemporaneously with the probiotic. To illustrate this embodiment, the active agent is administered for a definite period of time (for illustration, three times day on an every other day schedule for a ten week period of time); upon conclusion of this illustrative ten week period of time, administration of the active agent continues along with commencement of administration of the probiotic, with active agent administered on a less than daily schedule and probiotic dosing for a definite period of time that can be daily or on a less than daily schedule or, optionally, indefinitely.

In other embodiments herein, the active agent is administered prior to administration of a probiotic and/or prebiotic and is also administered contemporaneously with the probiotic and/or prebiotic. To illustrate this embodiment, the active agent is administered for a definite period of time (for illustration, three times day on an every other day schedule for a ten week period of time); upon conclusion of this illustrative ten week period of time, administration of the active agent continues along with commencement of administration of the probiotic and/or prebiotic, with active agent administered on a less than daily schedule and probiotic and/or prebiotic dosing for a definite period of time that can be daily or on a less than daily schedule or, optionally, indefinitely.

### Probiotic

In certain embodiments, the methods of the present invention may utilize a probiotic. Probiotics have been shown to inhibit pathogen adherence to colonic mucosa, increase immunoglobulin-A (IgA) secretion in Peyer's patches, increase immune activity inhibiting the release of anti-inflammatory cytokines and inhibiting pro-inflammatory cytokines. See Gionchetti P, Amadini C, Rizzello F, Venturi A, Palmonari V, Morselli C, et al. Probiotics—role in inflammatory bowel disease. Dig Liver Dis. 2002;34(Suppl 2):S58-62. Probiotics may also interfere with pathogen metabolism.

Advancing age may be associated with decreased bifidobacteria and increased Bacteroides species. It should also be noted that low fiber diets may actually alter the colonic microecology: it has been demonstrated in humans that wheat bran adversely changes the anerobic/aerobic bacterial ratios See Floch M H, Fuchs H M. Modification of stool content by increased bran intake. Am J Clin Nutr. 1978;31 (10 Suppl):S 185-9.This has implications for the application of probiotics as prophylaxis against bacteroides overgrowth and infection leading to inflammatory bowel disorder.

The probiotic may be, for example, lactic acid bacteria. Non-limiting examples of lactic acid bacteria suitable for use herein include strains of Streptococcus lactis, Streptococcus cremoris, Streptococcus diacetylactis, Streptococcus thermophilus, Lactobacillus bulgaricus, Lactobacillus acidophilus, Lactobacillus helveticus, Lactobacillus bifidus, Lactobacillus casei, Lactobacillus lactis, Lactobacillus plantarum, Lactobacillus rhamnosus, Lactobacillus delbruekii, Lactobacillus thermophilus, Lactobacillus fermentii, Lactobacillus salivarius, Bifidobacterium longum, Bifidobacterium infantis, Bifidobacterium bifidum, and Pediococcus cerevisiae, or mixtures thereof. In one embodiment the probiotic is Lactobacillus salivarius, Bifidobacterium infantis, or mixtures thereof. In another embodiment, the probiotic is Bifidobacterium infantis.

As a non-limiting example, strains of Lactobacillus salivarius isolated from resected and washed human gastrointestinal tract as described in WO 98/35014 are preferred. More preferred are the Lactobacillus salivarius strains that are designated UCC 1 and UCC 118, described as being deposited at the National Collections of Industrial and Marine Bacteria Ltd (NCIMB) on Nov. 27, 1996, and accorded the accession numbers NCIMB 40830 and 40829, respectively.

As another non-limiting example, the probiotic is a Bifidobacterium spp. For example, strains of Bifidobacterium spp. isolated from resected and washed human gastrointestinal tract as disclosed in WO 00/42168 may be used herein. One example is the Bifidobacterium infantis strain designated as UCC35624, described as being deposited at the National Collections of Industrial and Marine Bacteria Ltd (NCIMB) on Jan. 13, 1999, and accorded the accession number NCIMB 41003.

The compositions used herein may be given to an individual as part of a dose regimen which may be dependent upon the dosing format used in which the probiotic is incorporated. The composition can comprise at least one strain of a probiotic, alternatively the composition can comprise two strains of a probiotic that are the same or they can be different strains, alternatively the composition can comprise three strains of a probiotic. A single dose of the probiotic comprises at least about 10³ cfu of the probiotic, alternatively at least about 10⁴ cfu of the probiotic, alternatively 10⁵ cfu of the probiotic. For example, the unit dose provides the mammal being treated with probiotic at a level of from about 1×10⁵ colony forming units (cfu) per dose to about 1×10¹⁵ cfu per dose, alternatively from about 1×10⁷ cfu to about 1×10¹⁴ cfu per dose, alternatively a single dose of the probiotic comprises from about 10⁸ cfu to about 10¹² cfu per dose. As another example, the dose regimen may commence at a higher dose, followed by a lower maintenance dose. See, for example, U.S. application Ser. No. 12/056,702, filed Mar. 27, 2008.

Any of a variety of different dose forms may be appropriate for administration. For example, for oral administration, the unit dose, when provided as a capsule, tablet, or other typical oral dosage form may be swallowed directly. As another example, when provided as a sachet filled with the probiotic, the probiotic may be ingested directly, or mixed with milk, yogurt, or another liquid carrier material. Typically, as an example, a dose form such as a capsules may provide lower dosing amounts than sachets, as the size of the capsule, and its relative easy of ingestion, will limit the amount of the probiotic that can be filled therein.

### Prebiotic

In certain embodiments, the methods of the present invention may utilize a prebiotic and/or a fiber. As used herein, the term “prebiotic” includes substances or compounds that beneficially affect the host mammal by selectively promoting the growth and/or activity of one or more probiotic bacterial in the gastro-intestinal tract of the host mammal, thus maintaining normal health or improving health of the host. Typically, prebiotics are carbohydrates, (such as oligosaccharides), but the term “prebiotic” as used herein does not preclude non-carbohydrates. Many forms of “fiber” exhibit some level of prebiotic effect. Thus, there is considerable overlap between substances that can be classified as “prebiotics” and those that can be classified as “fibers”. Non-limiting examples of prebiotics suitable for use in the compositions and methods include psyllium, fructo-oligosaccharides, inulin, oligofructose, galacto-oligosaccharides, isomalto-oligosaccharides xylo-oligosaccharides, soy-oligosaccharides, gluco-oligosaccharides, mannan-oligosaccharides, arabinogalactan, arabinxylan, lacto sucrose, gluconannan, lactulose, polydextrose, oligodextran, gentioligosaccharide, pectic oligosaccharide, xanthan gum, gum arabic, hemicellulose, resistant starch and its derivatives, and mixtures and/or combinations thereof. The compositions can comprise from about 100 mg to about 100 g, alternatively from about 500 mg to about 50 g, and alternatively from about 1 g to about 40 g, of prebiotic, per day or on a less than daily schedule.

### Antibiotic

Current standard therapy using antibiotics alone may address acute attacks, but not thereafter the attack (e.g., symptoms may persist). As an example, in order to conform with the current standard of care, in certain embodiments, the methods and kits may optionally comprise administration of an antibiotic.

Antibiotics are commonly known and are selected by one of ordinary skill in the art. To illustrate, the antibioitic may be selected from the group consisting of metronidazole, cephalexin, ciprofloxacin, levofloxacin, moxifloxacin, gemifloxacin, balofloxacin, gatifloxacin, grepafloxacin, pazufloxacin, sparfloxacin, temafloxacin, enoxacin, fleroxacin, lomefloxacin, nadifloxacin, norfloxacin, ofloxacin, pefloxacin, rufloxacin, trovofloxacin, tosufloxacin, clindamycin, tetracycline, chloramphenicol, cefoxitin, cefmetazole, cefotetan, doxycycline, erythromycin, imipenem, meropenem, ticarcillin, pipercillin, mezocillin, tazobactam, ampicillin, and combinations thereof.

As illustrative guidelines, subjects presenting with inflammatory bowel disorder symptoms may be dosed a single type of antibiotic, while those presenting with more severe symptoms may be dosed a combination of antibiotics, including two, three, or even more distinct antiobiotics. For example, two distinct antibiotics may be prescribed for those with moderate symptoms, while triple therapy may be prescribed for those presenting with severe symptoms. In many of these moderate to severe cases, intravenous therapy may be appropriate, while typically oral dosing is also appropriate.

Dose level and frequency will be commonly understood in the art, and may be dependent upon the antibiotic employed. Merely as illustration, the methods and kits herein may optionally utilize a daily dose of from about 50 mg to about 6000 mg of antiobiotic, or from about 100 mg to about 2500 mg of antibiotic, or from about 250 mg to about 2000 mg of antibiotic. Daily dosing may be administered as a single dose, or divided into multiple doses such as twice daily, three times daily, or four times daily dosing.

To illustrate, the methods and kits herein may utilize administration of metronidazole (for example, FLAGYL). The metronidazole may optionally be administered orally in tablet form, optionally in immediate or sustained release forms. Other useful forms may include topical or intranvenous forms, or any other form that would be useful herein. A commonly used oral dose may include administration of from about 250 mg to about 750 mg of the metronidazole on a daily basis until antibiotic administration is complete.

As yet another illustration, the methods and kits herein may utilize administration of cephalexin (for example, KEFLEX, KEFTABS, or BIOCEF). The cephalexin may optionally be administered orally in tablet form, optionally in immediate or sustained release forms. Other useful forms may include powders for suspension, or any other form that would be useful herein. A commonly used oral dose may include administration of from about 250 mg to about 750 mg of the cephalexin on a daily basis until antibiotic administration is complete.

As yet another illustration, the methods and kits herein may utilize administration of doxycycline (for example, VIBRAMYCIN). The doxycycline may optionally be administered orally in tablet form, optionally in immediate or sustained release forms. Other useful forms may include suspensions, or any other form that would be useful herein. A commonly used oral dose may include administration of from about 50 mg to about 300 mg of the doxycycline on a daily basis until antibiotic administration is complete.

As yet another illustration, two or more discrete antibiotics may be utilized. For example, one may choose an antibiotic having anaerobic bacteria kill, and a different antibiotic having aerobic bacteria kill. For example, the methods and kits could utilize ciprofloxacin at a range of from about 250 mg per day to about 2000 mg per day along with metranidazole at a range of from about 250 mg per day to about 6000 mg per day.

In one embodiment, administration of the antibiotic is prior to the administration of the probiotic, prior to the administration of the active agent, or prior to the administration of the probiotic and the active agent; or prior to the administration of the probiotic and/or prebiotic and the active agent. In certain embodiments, administration of the antibiotic is prior to the administration of the active agent and the probiotic. For example, the antibiotic may be administered for a period of from 1 day to about 30 days following an acute attack of a inflammatory bowel disorder, or from 1 day to about 14 days, or from about 7 days to about 10 days. The active agent and the probiotic may also be administered, either contemporaneously or during different time periods.

The foregoing described dosage levels for active agent, probiotic, and antibiotic are based on typical human subjects (e.g., about a 55 to 65 kg subject). Wherein the present composition is used in other mammals, it may be necessary to modify the dosage. Modification of dosage based on the needs of the subject is well within the skill of the ordinary artisan. It is therefore understood that these dosage ranges are by way of example only, and that administration can be adjusted depending on various factors.

The specific dosage of the active agent, probiotic, and antibiotic, as well as the duration of treatment may be interdependent. The dosage and treatment regimen may also depend upon such factors as the specific active agent, probiotic, and antibiotic used, as applicable, the treatment indication, the efficacy of the agent used, the personal attributes of the subject (such as, for example, weight, age, gender, and medical condition of the subject), and compliance with the treatment regimen.

### Kits

In yet another embodiment, the invention is directed to kits, wherein the kits are for use in a regimen for treating, inhibiting, and/or maintaining remission of a gastrointestinal disorder, said regimen comprising orally administering a first dose of a pharmaceutical composition comprising from about 1 mg to about 8000 mg of an active agent and continuing said treatment, inhibition and or maintaining remission by orally administering an additional dose on a less than a daily schedule of an active agent, said kit comprising:
a. one dose of an active agent; and
b. from about one to about eighty-four additional doses of an active agent.

In yet another embodiment, the invention is directed to kits, wherein the kits are for use in a regimen for treating, inhibiting, and/or maintaining remission of a gastrointestinal disorder comprising: commencing treatment by orally administering a dose on an every other day schedule of a pharmaceutical composition comprising from about 1 mg to about 8000 mg of an active agent, and said kit further comprising from about one to about eighty-four additional doses of said active agent given on an every other day schedule.

The kits may optionally comprises, a probiotic, a prebiotic and/or an antibiotic. The kits may comprise from about 1 to about 100 doses of probiotics, or from about 7 to about 60, or from about 7 to about 30 doses of probiotics. The kits may comprise from about 1 to about 100 doses of prebiotics, or from about 7 to about 60, or from about 7 to about 30 doses of prebiotics.

The active agents, probiotics, prebiotics, and antibiotics, including various embodiments or selections thereof, are as described herein.

In one embodiment, the kits comprise one or more discrete compositions comprising the active agent and one or more discrete compositions comprising the probiotic. For example, the kit may comprise a less than daily dose, weekly, monthly, or other periodic dose of the active agent and the probiotic. As an illustrative example, a kit comprising a weekly dose may comprise 7 discrete compositions comprising the probiotic (7 daily doses), 1 discreet dose comprising the first dose of the active agent and 18 discrete compositions comprising additional dose of the active agent (3 doses taken in one day, each complete every other day dose comprised of six discrete compositions). As another example, a kit comprising a monthly dose may comprise 30 discrete compositions comprising the probiotic (30 daily doses), 1 discreet dose comprising the first dose of the active agent and 84 discrete compositions comprising the active agent administered on an every other day schedule. As another example, a kit comprising a monthly dose may comprise 30 discrete compositions comprising the probiotic (30 daily doses), 30 discrete compositions comprising the prebiotic (30 daily doses), 1 discreet dose comprising the first dose of the active agent and 84 discrete compositions comprising the active agent administered on an every other day schedule. These kits may optionally comprise the antibiotic, for example in accordance with the number of doses of antibiotic intended for use. For example, wherein it is desired that 10 daily doses of antibiotic is administered prior to administration of the active agent and prior to the probiotic, the kit may optionally contain 10 discrete compositions comprising the antibiotic. Any of a variety of combinations of types and numbers of discrete compositions will be selected by those of ordinary skill in the art.

In certain embodiments, the kits are configured to facilitate dosing compliance. For example, the kits may be particularly advantageous for the purpose of ensuring that the subject is receiving administration of all components (for example, probiotic, active agent, prebiotic, antibiotic, first dose of active agent, additional dose of active agent) on the appropriately prescribed schedule. Blister cards or other containing devices appropriately configured may be particularly suitable for clearly illustrating sequence or timing of administration of the various components. Various configurations will be well known to the ordinarily skilled artisan in view of the present specification.

The kits of the present invention may comprise one or more of the active agent, one or more of the probiotic, one or more of the prebiotic, and antibiotic, optionally together with information which informs a user of the kit, by words, pictures, and/or the like, that use of the kit will provide one or more general health and/or general physiological benefits including, but not limited to, gastrointestinal health benefits (for example relief from, prevention of, treatment of and/or inhibition of a inflammatory bowel disorder), and/or general active agent benefits. Such information need not utilize the actual words used herein, for example, “inflammatory bowel disorder”, “IBD”, “IBS”, Inflammatory IBS”, disease”, “condition”, or “gastrointestinal”, but rather use of words, pictures, symbols, and the like conveying the same or similar meaning are contemplated within the scope of this invention.

In one embodiment, the information is printed on a container holding the composition, e.g., a box, card (e.g., a blister card), or other containing device. These preferred kits may be in the form of one containing device containing the composition, or may be obtained as a plurality of devices each containing the composition. For example, the kits may be obtained as one card, or cases of four, six, seven (e.g., a weekly supply), or eight cards co-packaged together. Additionally, monthly or other types of kits may be obtained.

### Method of Manufacture

The compositions and various components used in the present invention may be manufactured in accordance with known methods.

### Experimental Study

A study was conducted to determine the effect of Olsalazine on dextran sulfate salt (DSS) induced chronic ulcerative colitis in male Swiss-Webster mice. Groups of 20 male Swiss-Webster mice were allowed ad libitum access to Olsalazine qd beginning on day −3 and then continued daily (qd) or every two days (q2d) starting on day 0 and continuing through day 14. Dipentum®, Olsalazine Sodium is a sodium salt of salicylate, disodium 3,3′-azobis(6-hydroxybenzoate) and when exposed to colonic bacteria converts to mesalamine. In this animal model, male Swiss-Webster mice are exposed to DSS (3% days 0-14) to induce inflammation and gland loss with erosion in the colon.

### Materials and Methods

### Test Article Identification and Preparation

Dextran sulfate, molecular weight 40-50,000 (sodium salt, Spectrum Chemicals, New Brunswick, N.J., Lot#RA1107) was stored at room temperature until added to appropriate volumes of tap water to prepare sufficient 3% DSS solutions. Prepared solutions were stored at 4° C. and added to water bottles daily. Every 3rd day, water in all bottles was discarded and replaced with fresh DSS preparations or tap water (normal controls). Dipentum® (Olsalazine Sodium) was received from the sponsor in hard gelatin capsule form. Capsules were received and each capsule contained 250 mg. On study day −3, 51 capsules were opened (˜12.75 grams) and the contents of each were consolidated into a labeled vial. Appropriate amounts of the beige-orange compound was then added to 250 ml water bottles so that each bottle would contain ˜0.83 mg/ml Olsalazine suspension in the water.

### Test System Identification

Male Swiss-Webster mice weighing approximately 22-29 grams on day (−)3 were obtained from Harlan, Inc (Indianapolis, Ind.). Animals were identified by color-coded dots at the base of the tail delineating group and animal number. After randomization, all cages were labeled with protocol number, group and animal numbers with appropriate color-coding.

### Environment and Husbandry

Upon arrival, animals were housed 5/cage in shoe-box polycarbonate cages with wire tops, wood chip bedding and suspended food and water bottles. The cages conformed to the guidelines cited in the Guide for the Care and Use of Laboratory Animals (8) and the applicable standard operating procedures of the University of Colorado vivarium. Animals were acclimated for 7 days prior to being placed on study. An attending Veterinarian was on site or on call during the live phase of the study.

During the acclimation and study periods, animals were housed in a laboratory environment with temperatures ranging between 67-76° F. and relative humidity between 30-70%. Automatic timers provided 12 hours of light and 12 hours of dark. Animals were allowed access ad libitum to Harlan Teklad Rodent Chow and fresh municipal tap water.

No concurrent medications were given. Animal care including room, cage and equipment sanitation conformed to the guidelines cited in the Guide for the Care and Use of Laboratory Animals.

### Experimental Design

Animals (5/group/normal control, 20/group for diseased treated), housed 5/cage, were given 3.0% DSS in their water from test days 0-14. Olsalazine treatment was initiated on day −3 and continued daily through day 14, or on days −3, −2, −1, 0, 2, 4, 6, 8, 10, 12, and 14 for the q2d group. Animals were terminated on day 14.
Experimental groups were as follows:

| Group | Inducer | Treatment | Treatment Dose | # of Animals |
|---|---|---|---|---|
| 1 | Normal | Vehicle, qd | ad libitum | 5 |
| | Control | | | |
| 2 | 3% DSS | Vehicle, qd | ad libitum | 20 |
| 3 | 3% DSS | Olsalazine, qd | ad libitum | 20 |
| 4 | 3% DSS | Olsalazine, | ad libitum | 20 |
| | | q2d | | |

### Observations, Measurements, and Specimens

Animals were weighed on study days −3 to 14. On day 14 mice were weighed, euthanized, and the entire colon was removed and the length determined. The colons were scored for gross changes using the following criteria:

0=normal, no blood observed

1=semi-solid stool

2=semi-solid to fluid stool

3=semi-solid with definite evidence of blood, bloody fluid or no content

Only animals that survived to study termination were included in the analysis of terminal colon lengths and scores.

### Morphologic Pathology Methods

For each surviving animal, the entire colon (proximal and distal) was trimmed into 8 equally spaced pieces for processing and embedding. Sections were stained with hemotoxylin and eosin (H&E). For each colon section, submucosal edema was quantified by measuring the distance (mm) from the muscularis mucosa to the internal border of the outer muscle layer in representative areas of edema. The extent of inflammation (foamy macrophages, lymphocytes and polymorphonuclear cell infiltrate) was assigned severity scores according to the following ranking:

0=Normal

1=Minimal

2=Mild

3=Moderate

4=Marked

5=Severe

The parameters reflecting epithelial cell loss/damage were scored individually using a percent area involved scoring method:

0=None

1=1-10% of the mucosa affected

2=11-25% of the mucosa affected

3=26-50% of the mucosa affected

4=51-75% of the mucosa affected

5=76-100% of the mucosa affected

Parameters that were scored using the above percent involvement scale include:
1) Colon glandular loss—includes crypt epithelial as well as remaining gland epithelial loss.
2) Colon erosion—reflects loss of surface epithelium and generally is associated with mucosal hemorrhage (seen clinically and at necropsy).

Mucosal epithelial hyperplasia (basophilia, mitotic figures, multi-layered on basement membranes, absence of goblet cells) was scored 0-5 based on the following criteria

0=Normal

1=Minimal—small foci generally adjacent to the inflammatory changes

2=Mild—11-25% of mucosa affected

3=Moderate—26-50% of mucosa affected

4=Marked—51-100% of mucosa affected

5=Severe—51-100% of mucosa affected plus papillary proliferation into lumen

Mucosal thickness (an indicator of proliferative changes or edematous inflammatory mucosal expansion) was measured by placing an ocular micrometer at the base of the glands and the overlying surface epithelium in a non-tangential area of section representative of the thickest areas of mucosa. The scores for proximal and distal colon were averaged to determine a score for the entire colon for each parameter evaluated. The 3 scored parameters (i.e., inflammation, glandular loss, and surface epithelial erosion) were combined to arrive at a sum of overall histopathology scores for the colon. These summations indicate the overall damage in the entire colon and would have a maximum severity score of 15.

### Statistical Analysis

From numerical data generated, arithmetic means and standard errors were calculated. Statistical analyses for clinical data, necropsy assessments, and histopathologic evaluations were conducted on all animals surviving to scheduled termination using a two-tailed Student!s t-test. Comparisons were performed between DSS treatments and the disease control group with significance set at p″0.05. Results were further analyzed using a one-way analysis of variance (ANOVA) with significance set at p″0.05. Individual group comparisons were made using the appropriate multiple comparison post-test (Dunnett for measurements and Dunn!s for scored parameters). LD50 calculations were done using Prism4 software to create survival curves for each group by the Kaplan/Meier product limit method and comparing the survival curves using the logrank test. Significance was set at p″0.05.

### Results

### Live Phase and Necropsy Parameters

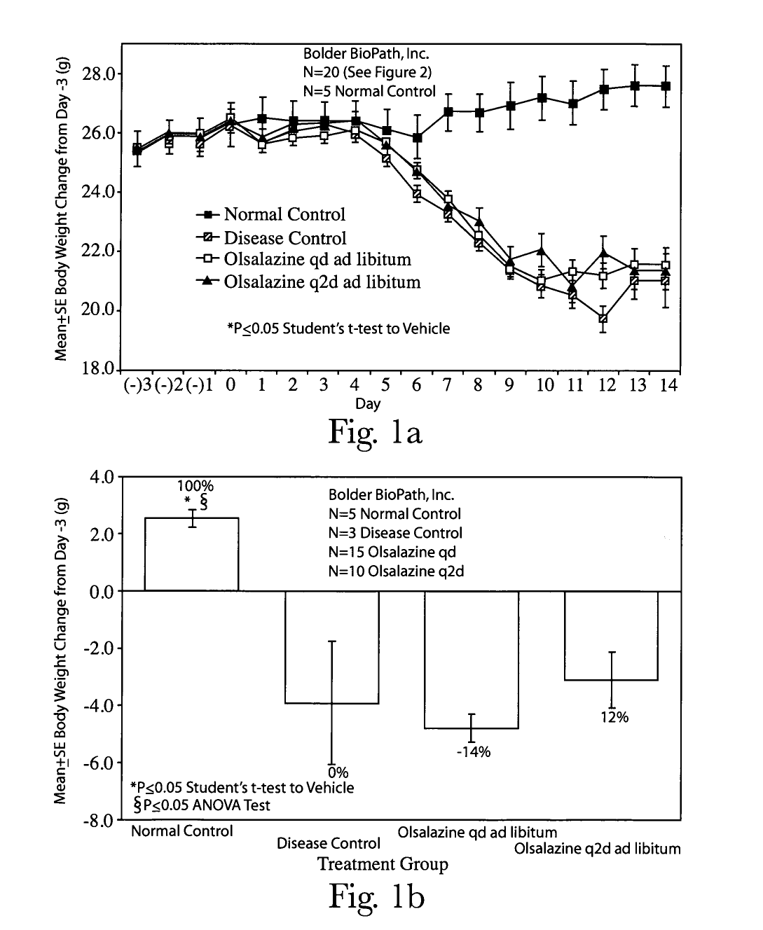

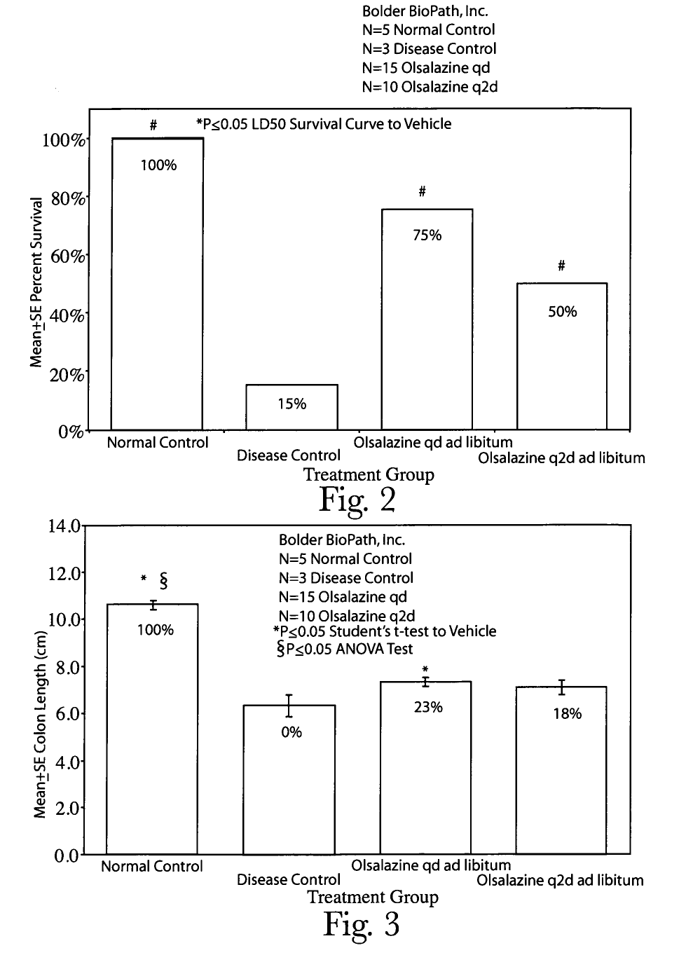

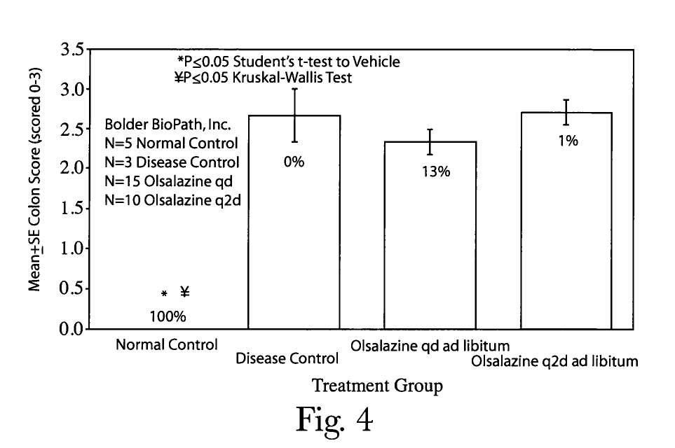

All animals in the normal control group survived the duration of the study (FIG. 2). Untreated normal control animals showed a mild increase (2.53 g) in body weight over the course of the study (FIG. 1a, FIG. 1b). Mean colon length for normal mice was 10.60 mm (FIG. 3). There were no clinical or necropsy alterations at termination day. There were 17 deaths in the vehicle treated control group (DSS control), with 15% survival to necropsy day (FIG. 2). Disease control mice showed a mean body weight loss of 3.90 g over the course of the study (FIG. 1a, FIG. 1b). The disease control group had significantly shorter colon lengths at termination day (6.33 mm), with mild to moderate gross pathology scores of the colon as indicated by colon content scores ranging from 2-3 (mean 2.67) (FIG. 3, FIG. 4).

Treatment with Olsalazine ad libitum, every day resulted in 75% (significant) survival to necropsy day (FIG. 2). There was no beneficial effect on body weight loss, animals losing a mean of 4.78 g at study end (FIG. 1a, FIG. 1b). Decreased colon length for treated animals was significantly inhibited by 23% (7.33 mm mean length), with colon content scores ranging from slight to moderate for a mean score of 2.33 (FIG. 3, FIG. 4). Treatment with Olsalazine ad libitum, every other day resulted in 50% (significant) survival to necropsy day (FIG. 2). There was no beneficial effect on body weight loss, animals losing a mean of 3.10 g at study end (FIG. 1a, FIG. 1b). Colon length for treated animals was mildly inhibited by 18% (7.10 mm mean length), with colon content scores ranging from mild to moderate for a mean score of 2.70 (FIG. 3, FIG. 4).

### Morphologic Pathology

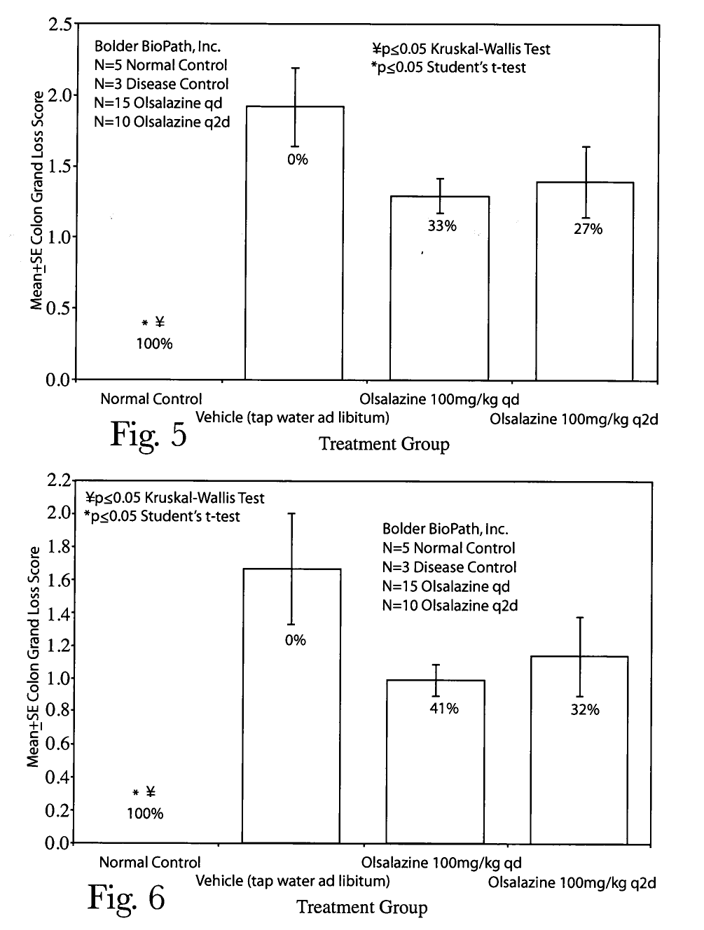

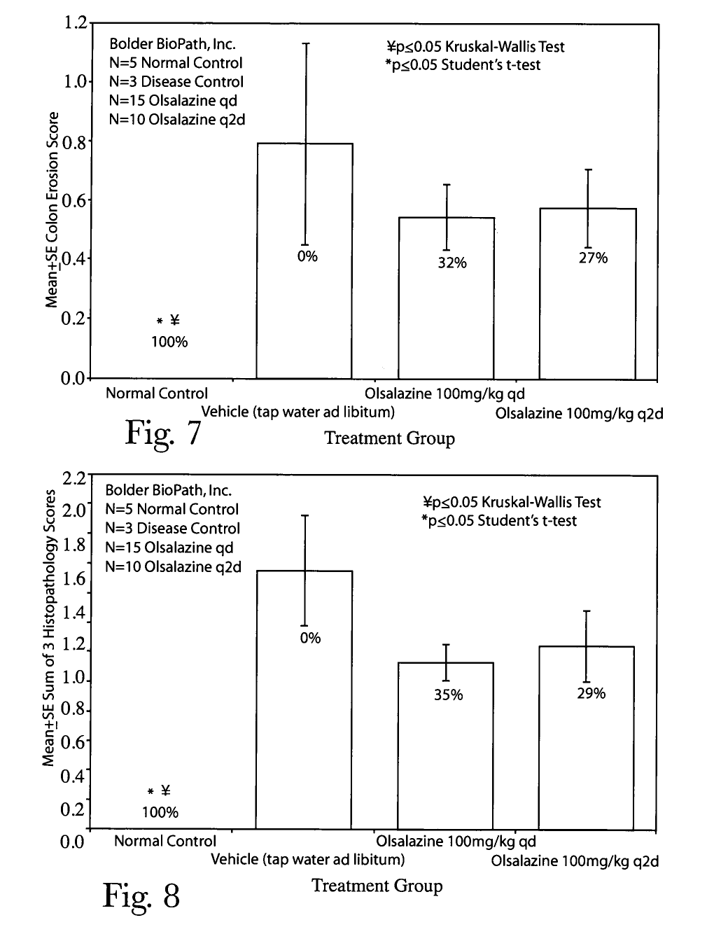

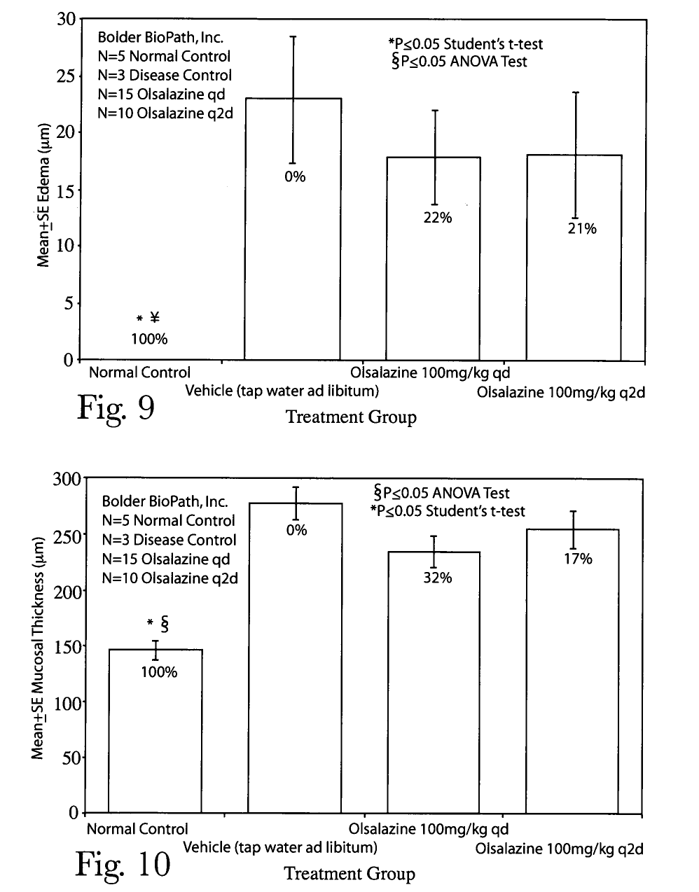

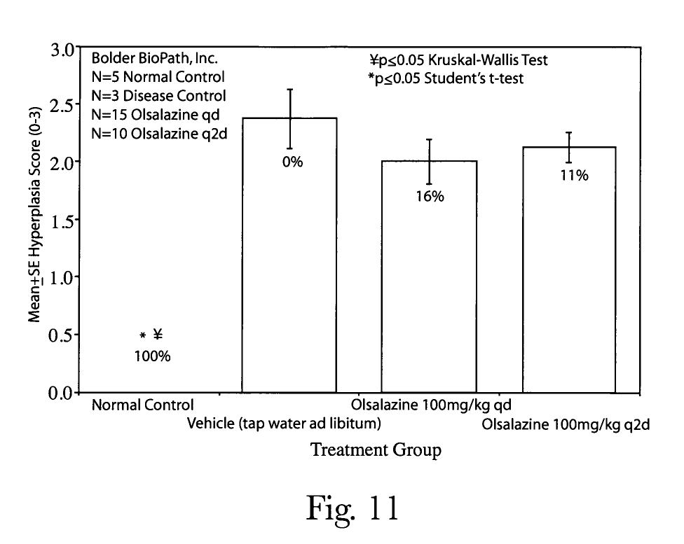

Three surviving vehicle treated control mice (DSS control) exposed to 3% DSS had minimal to severe colitis characterized by inflammation (accumulation of foamy macrophages and neutrophils) gland loss and erosion (FIG. 5, FIG. 6, FIG. 7, FIG. 8, FIG. 9). Mucosal hyperplasia was less common but when present was minimal to marked and mucosal thickness was increased approximately 2 times that of the normal controls (FIG. 10, FIG. 11). The normal control animals in this study showed no signs of colitis. Treatment of animals with Olsalazine ad libitum, qd had mild (non significant) beneficial effects on colon edema with 22% inhibition of histopathologic scores (FIG. 9). Treatment resulted in moderate inhibition of colon erosion (32%) and significant inhibition of colon inflammation and gland loss (33%, 32% respectively) for a sum colon histopathologic score of 35% (non significant) (FIG. 5, FIG. 6, FIG. 7, FIG. 8). An increase of mucosal thickness was moderately inhibited by 32% (FIG. 10). Hyperplasia scores ranged from minimal to moderate for a mean score that was inhibited by 16% as compared to disease control treated mice (FIG. 11). Treatment of animals with Olsalazine ad libitum, q2d had mild (non-significant) inhibition on colon edema, inflammation, and erosion (21%, 27%, 27%,respectively) (FIG. 5, FIG. 7, FIG. 9). Treatment resulted in moderate inhibition of colon gland loss (32%) for a sum colon histopathologic score of 29% (non significant) (FIG. 6, FIG. 8). Mucosal hyperplasia was less common but when present was minimal to mild for a mean score inhibited by 11% and mucosal thickness was inhibited by 17% compared to disease controls (FIG. 10, FIG. 11).

### Discussion/Conclusions

Results of this study indicated that treatments of Olsalazine ad libitum (qd or q2d), initiated 3 days prior to exposure to DSS and continued for 14 days, had excellent, dose responsive (75% for qd, 50% for q2d vs 15% in disease controls) beneficial effects on survivability. Most of the conclusions should be drawn from the clinical survival data. The body weight change, colon length/score, and histopathology data underestimates the efficacy because a large number of the disease control animals, presumably with the most severe lesions, died. Significance for these parameters, when achieved, is remarkable but where it wasn't achieved-the caveat mentioned above should be applied.

Treatment with Olsalazine ad libitum, every day resulted in 75% (significant) survival to necropsy day. There was no beneficial effect on body weight loss, animals losing a mean of 4.78 g at study end. Decreased colon length for treated animals was significantly inhibited by 23% (7.33 mm mean length), with colon content scores ranging from slight to moderate for a mean score of 2.33. Treatment with Olsalazine ad libitum, every other day resulted in 50% (significant) survival to necropsy day. There was no beneficial effect on body weight loss, animals losing a mean of 3.10 g at study end. Colon length for treated animals was mildly inhibited by 18% (7.10 mm mean length), with colon content scores ranging from mild to moderate for a mean score of 2.70.

Three surviving vehicle treated control mice (DSS control) exposed to 3% DSS had minimal to severe colitis characterized by inflammation (accumulation of foamy macrophages and neutrophils) gland loss and erosion. Mucosal hyperplasia was less common but when present was minimal to marked and mucosal thickness was increased approximately 2 times that of the normal controls. The normal control animals in this study showed no signs of colitis.

Treatment of animals with Olsalazine ad libitum, qd had mild (non significant) beneficial effects on colon edema with 22% inhibition of histopathologic scores. Treatment resulted in moderate inhibition of colon erosion (32%) and significant inhibition of colon inflammation and gland loss (33%, 32% respectively) for a sum colon histopathologic score of 35% (non significant). An increase of mucosal thickness was moderately inhibited by 32%. Hyperplasia scores ranged from minimal to moderate for a mean score that was inhibited by 16% as compared to disease control treated mice. Treatment of animals with Olsalazine ad libitum, q2d had mild (non significant) inhibition on colon edema, inflammation, and erosion (21%, 27%, 27%, respectively). Treatment resulted in moderate inhibition of colon gland loss (32%) for a sum colon histopathologic score of 29% (non significant). Mucosal hyperplasia was less common but when present was minimal to mild for a mean score inhibited by 11% and mucosal thickness was inhibited by 17% compared to disease controls.

### Examples

The following examples further describe and demonstrate embodiments within the scope of the invention. The examples are given solely for the purpose of illustration and are not to be construed as limitations of the present invention, as many variations thereof are possible without departing from the spirit and scope of the invention.

### Example I

A 60 kg woman is diagnosed with irritable bowel syndrome and the colonoscopy demonstrates inflammation around the nerves in the wall of the colon which control peristalsis. The fecal surrogate markers of inflammation are also raised. She is prescribed a pharmaceutical composition comprising 2400 milligrams delayed-release mesalamine, to be taken on an every other day regimen. The probiotic, Bifidobacterium infantis 35624 at a dose of 10⁸ CFU is administered on the mesalamine-free days also on an every other day regimen. A repeat biopsy of the colonic wall will be taken 6 months later to determine if there is a decrease in the inflammation around the nerves which control peristalsis.

### Example II

A 65 kg woman is diagnosed with irritable bowel syndrome and the colonoscopy demonstrates inflammation around the nerves in the wall of the colon which control peristalsis. The fecal surrogate markers of inflammation are also raised. She is prescribed a pharmaceutical composition comprising 4800 milligrams delayed-release mesalamine, taken on an every other day regimen to be taken for 2 weeks only. On the 15^{th} day (after completing the mesalamine) the patient commences every day administration of the probiotic, Bifidobacterium infantis 35624 at a dose of 10⁸ CFU. A repeat biopsy of the colonic wall will be taken 6 months later to determine if there is a decrease in the inflammation around the nerves which control peristalsis.

### Example III

A 75 kg man has been using numerous OTC medicines multiple times per day for what his general physician calls a “spastic colon” (prn antispasmodics, anti-diarrheals, laxatives and analgesics). A colonoscopy shows inflammation in the mucosa and wall of the colon. There is also a raised level of inflammatory surrogate markers in the fecal effluent specimens. The patient is prescribed a pharmaceutical composition to be taken on every other day regimen for 1 month. Each unit dose of the pharmaceutical composition comprises 4800 milligrams of delayed-release mesalamine. The patient takes a unit dose of the pharmaceutical composition once every other day every morning. At the end of 1 month, the patient starts every day probiotic, Bifidobacterium infantis 35624, at a dose of 10⁸ CFU. A follow-up colonic biopsy will be taken 6 months later and determine if there is a decrease in the degree of inflammation. A repeat laboratory evaluation of fecal inflammatory markers will also be evaluated to see if it also shows a reduction at 6 months.

### Example IV

A 75 kg man is diagnosed with inflammatory irritable bowel syndrome by colonoscopy which demonstrates inflammation in the wall of his colon. The patient is prescribed 4800 milligrams of delayed-release mesalamine to be taken on every other day regimen for 1 month along with the probiotic, Bifidobacterium infantis 35624 at a dose of 10¹⁰ CFU also administered on alternate days as the delayed-release mesalamine. At 1 month the patient goes onto an every other day regime dosing of 2400 milligrams of the delayed-release mesalamine and every day dosing of the Bifidobacterium infantis 35624, 10¹⁰ CFU.

### Example V

A 67 kg woman is diagnosed with inflammatory irritable bowel syndrome by colonoscopy which demonstrates inflammation in the wall of the colon. The patient is prescribed a pharmaceutical composition to be taken on an every other day regimen and a probiotic, also to be taken on every other day regimen. Each unit dose of the pharmaceutical composition comprises 2400 milligrams of delayed-release mesalamine. Each unit dose of probiotic to be taken every other day comprises 10¹² CFU Bifidobacterium infantis 35624.

### Example VI

A 78 kg man is diagnosed with microscopic colitis by colonoscopy which demonstrates biopsy proven inflammation in the wall of the colon. The patient is prescribed a pharmaceutical composition to be taken on every other day regimen for 1 month and a probiotic, also to be taken on every other day regimen for a month. Each unit dose of the pharmaceutical composition comprises 4800 milligrams of delayed-release mesalamine. Each unit dose of probiotic to be taken on every other day comprises 10¹² CFU Bifidobacterium infantis 35624. After 1 month, the patient is then prescribed 2400 milligrams of delayed-release mesalamine on every other day regimen and 10⁸ CFU Bifidobacterium infantis 35624 on every day. A repeat biopsy of the colonic wall will be taken 6 months later to determine if there is a decrease of the inflammation around the nerves which control peristatlsis.

### Example VII

A 45 kg woman is diagnosed with diverticular disease by colonoscopy which demonstrates inflammation in the wall of the colon. The patient is prescribed a pharmaceutical composition to be taken on every other day regime for 1 month and a probiotic, also to be taken on every other day for a month. Each unit dose of the pharmaceutical composition comprises 4800 milligrams of delayed-release mesalamine. Each unit dose of probiotic to be taken on every other day comprises 10¹² CFU Bifidobacterium infantis 35624. After 1 month, the patient is then prescribed 2400 milligrams of delayed-release mesalamine on every other day regimen and 10⁸ CFU Bifidobacterium infantis 35624 on every other day regimen. A repeat biopsy of the colonic wall will be taken 6 months later to determine if there is a decrease of the inflammation around the nerves.

### Example VIII

A 76 kg woman is diagnosed having an exacerbation of her inflammatory irritable bowel syndrome. Previous work-up by colonoscopy had demonstrated inflammation in the wall of the colon. The patient is prescribed a pharmaceutical composition to be taken daily and a probiotic, also to be taken on a daily dosing regimen for the acute flare of IBS for up to 1 month. Each unit dose of the pharmaceutical composition comprises 4800 milligrams of delayed-release mesalamine. Each unit dose of probiotic to be taken comprises 10¹² CFU Bifidobacterium infantis 35624. After the flare subsides, the patient is administered 1600 milligrams of delayed-release mesalamine on every other day regimen for the maintenance of remission of inflammatory irritable bowel syndrome symptoms.

### Example IX

A 67 kg woman is diagnosed with indeterminant colitis by colonoscopy which demonstrates micro-inflammation in the wall of the colon. The patient is prescribed a pharmaceutical composition to be taken on an every other day regimen for the duration of the colitis flare. Each unit dose of the pharmaceutical composition comprises 4800 milligrams of delayed-release mesalamine. After the flare-up has subsided the patient is then administered a probiotic to be taken on every other day regimen comprising 10¹² CFU Bifidobacterium infantis 35624.

### Example X

A 72 kg woman is diagnosed with collagenous colitis by colonoscopy which demonstrates inflammation in the wall of the colon. The patient is prescribed a regimen consisting of a pharmaceutical composition to be taken on every other day and a probiotic, also to be taken on an every other day dosing regimen for 2 weeks. Each unit dose of the pharmaceutical composition comprises 4800 milligrams of delayed-release mesalamine. Each unit dose of probiotic to be taken comprises 10¹² CFU Bifidobacterium infantis 35624. After 2 weeks the regimen changes to delayed-release mesalamine administered at 1600 milligrams and 10⁸ CFU Bifidobacterium infantis 35624 on every other day regimen.

### Example XI

An 87 kg man is diagnosed with inflammatory irritable bowel syndrome by colonoscopy which demonstrates inflammation in the wall of the colon. The patient is prescribed a pharmaceutical composition to be taken on an every other day regimen and a probiotic, also to be taken on an every other day dosing regimen. Each unit dose of the pharmaceutical composition comprises 1600 milligrams of delayed-release mesalamine. Each unit dose of probiotic comprises 10⁸ CFU Bifidobacterium infantis 35624.

### Example XII

A 57 kg woman is diagnosed with irritable bowel syndrome and the colonoscopy demonstrates inflammation around the nerves in the wall of the colon which control peristalsis. The fecal surrogate markers of inflammation are also raised. She is prescribed an oral first dose of a pharmaceutical composition comprising 4000 mg of delayed-release mesalamine, to be taken daily for 1 week. After 1 week, the delayed-release mesalamine is then administered orally at a dose of 2000 mg on every other day regimen. A repeat biopsy of the colonic wall will be taken 6 months later to determine if there is a decrease of the inflammation around the nerves which control peristalsis.

### Example XIII

A 84 kg male is diagnosed with irritable bowel syndrome and the colonoscopy demonstrates inflammation around the nerves in the wall of the colon which control peristalsis. The fecal surrogate markers of inflammation are also raised. He is prescribed a loading dose of a pharmaceutical composition comprising 5000 mg delayed-release mesalamine to be taken every other day for 2 weeks only. On the 15^{th} day (after completing the mesalamine) the patient then commences every other day oral administration of the probiotic, Bifidobacterium infantis 35624 at a dose of 10⁸ CFU as well as prebiotic given on the same day (every other day). A repeat biopsy of the colonic wall will be taken 6 months later to determine if there is a decrease of the inflammation around the nerves which control peristalsis.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for treating, inhibiting, and/or maintaining remission of a gastrointestinal disorder comprising: commencing treatment by orally administering a first dose of a pharmaceutical composition comprising from about 1 mg to about 8000 mg of an active agent and continuing said treatment, inhibition and or maintenance by orally administering an additional dose on a less than a daily schedule of an active agent.

2. The method of claim 1, wherein said less than daily schedule is selected from the group consisting of every other day, once weekly, twice weekly, once every two weeks, once monthly, once every other month, and combinations thereof.

3. The method of claim 2, wherein said less than daily schedule is every other day.

4. The method of claim 1, wherein said active agent is selected from a salicyclic acid derivative.

5. The method of claim 1, wherein said active agent is selected from the group consisting of aminosalicylate, sulfasalazine, 5-aminosalicylic acid, 4-aminosalicylic acid, benzalazine, dihydrochloride salt, olsalazine, balsalazide, bismuth subsalicylate, and mixtures thereof.

6. The method of claim 1, wherein said first dose is from about 50 mg to about 7000 mg of said active agent.

7. The method of claim 1, wherein said first dose is from about 200 mg to about 4800 mg of said active agent.

8. The method of claim 1, wherein said first dose is from about 1000 mg to about 4800 mg of said active agent agent.

9. The method of claim 1, wherein said additional dose is from about 200 mg to about 4800 mg of said active agent.

10. The method of claim 1, wherein said additional dose is from about 400 mg to about 4800 mg of said active agent.

11. The method of claim 1, wherein said method further comprising administration of a probiotic; wherein said probiotic is a Lactobacillus spp., Bifidobacterium spp., and mixtures thereof.

12. The method of claim 11, wherein said Bifidobacterium spp. comprises bacteria selected from the group consisting of Bifidobacterium longum, Bifidobacterium infantis, Bifidobacterium bifidum, and mixtures thereof.

13. The method of claim 12, wherein the Bifidobacterium spp. comprises Bifidobacterium infantis.

14. The method of claim 11 wherein a single dose of the probiotic comprises at least about 10³ cfu of the probiotic.

15. The method of claim 11, wherein a single dose of the probiotic comprises from about 10⁵ cfu to about 10¹⁵ cfu of the probiotic.

16. The method of claim 1, wherein said gastrointestinal disorder is selected from the group consisting of inflammatory bowel disease, Crohn's disease, ulcerative colitis, inflammatory bowel disorder, irritable bowel syndrome, irritable bowel syndrome-diarrhea, irritable bowel syndrome-constipation, irritable bowel syndrome-mixed, irritable bowel syndrome-alternating, dyspepsia, gastro-esophageal reflux, diverticulitis, diverticular disease, gastroparesis, microscopic colitis, lymphocytic colitis, collagenous colitis, indeterminant colitis, eosinophilic esophagitis, HIV-associated diarrhea, antibiotic-associated colitis, clostridium difficile-associated diarrhea, pseudo-membranous colitis, diarrhea associated with immunodeficiency disorders, small bowel overgrowth syndrome, celiac disease, Whipple's disease, CMV-associated colitis, Behcet's syndrome, and combinations thereof.

17. The method of claim 1, wherein said inflammatory bowel disorder is selected from the group consisting of inflammatory irritable bowel syndrome, irritable bowel syndrome-diarrhea, irritable bowel syndrome-constipation, irritable bowel syndrome-mixed, irritable bowel syndrome-alternating, dyspepsia, microscopic colitis, lymphocytic colitis, collagenous colitis, indeterminant colitis, celiac disease, and combinations thereof.

18. A method for treating, inhibiting, and/or maintaining remission of a gastrointestinal disorder comprising: commencing treatment by orally administering a pharmaceutical composition comprising an active agent on an every other day schedule said composition comprising from about 1 mg to about 8000 mg of said active agent.

19. The method of claim 13, wherein said active agent is selected from a salicyclic acid derivative.

20. The method of claim 13, wherein said active agent is selected from the group consisting of aminosalicylate, sulfasalazine, 5-aminosalicylic acid, 4-aminosalicylic acid, benzalazine, dihydrochloride salt, olsalazine, balsalazide, bismuth subsalicylate, and mixtures thereof.

21. The method of claim 18, comprising from about 200 mg to about 4800 mg of said active agent.

22. The method of claim 18, comprising from about 1000 mg to about 4800 mg of said active agent.

23. The method of claim 18, comprising from about 1500 mg to about 4800 mg of said active agent.

24. The method of claim 18, wherein said method further comprising administration of a probiotic; wherein said probiotic is a Lactobacillus spp., Bifidobacterium spp., or combinations thereof.

25. The method of claim 18, wherein said method further comprising administration of a prebiotic.

26. The method of claim 18, wherein said gastrointestinal disorder is selected from the group consisting of inflammatory bowel disease, Crohn's disease, ulcerative colitis, inflammatory bowel disorder, irritable bowel syndrome, irritable bowel syndrome-diarrhea, irritable bowel syndrome-constipation, irritable bowel syndrome-mixed, irritable bowel syndrome-alternating, dyspepsia, gastro-esophageal reflux, diverticulitis, diverticular disease, gastroparesis, microscopic colitis, lymphocytic colitis, collagenous colitis, indeterminant colitis, eosinophilic esophagitis, HIV-associated diarrhea, antibiotic-associated colitis, clostridium difficile-associated diarrhea, pseudo-membranous colitis, diarrhea associated with immunodeficiency disorders, small bowel overgrowth syndrome, celiac disease, Whipple's disease, CMV-associated colitis, Behcet's syndrome, and combinations thereof.

27. A kit for use in a regimen for treating, inhibiting, and/or maintaining remission of a gastrointestinal disorder, said regimen comprising orally administering a first dose of a pharmaceutical composition comprising from about 1 mg to about 8000 mg of an active agent and continuing said treatment, inhibition and or maintaining remission by orally administering an additional dose on a less than a daily schedule of an active agent, said kit comprising:
a. one dose of an active agent; and
b. from about one to about eighty-four additional doses of an active agent.

28. The kit of claim 27, further comprising a means for having said first dose and said additional doses arranged in a way as to facilitate compliance with the regimen.

29. The kit of claim 28, wherein the means is a card having arranged thereupon said first dose and said additional dose in their intended use.

30. The kit of claim 27, further comprising from about 7 to about 100 daily doses of a probiotic.

31. The kit of claim 27, further comprising from about 7 to about 100 daily doses of a prebiotic.

32. The method of claim 27, further comprising administering an antibiotic.

33. The kit of claim 27, wherein said gastrointestinal disorder is selected from the group consisting of inflammatory bowel disease, Crohn's disease, ulcerative colitis, inflammatory bowel disorder, irritable bowel syndrome, irritable bowel syndrome-diarrhea, irritable bowel syndrome-constipation, irritable bowel syndrome-mixed, irritable bowel syndrome-alternating, dyspepsia, gastro-esophageal reflux, diverticulitis, diverticular disease, gastroparesis, microscopic colitis, lymphocytic colitis, collagenous colitis, indeterminant colitis, eosinophilic esophagitis, HIV-associated diarrhea, antibiotic-associated colitis, clostridium difficile-associated diarrhea, pseudo-membranous colitis, diarrhea associated with immunodeficiency disorders, small bowel overgrowth syndrome, celiac disease, Whipple's disease, CMV-associated colitis, Behcet's syndrome, and combinations thereof.

34. A kit for use in a regimen for treating, inhibiting, and/or maintaining remission of a gastrointestinal disorder comprising: commencing treatment by orally administering a dose on an every other day schedule of a pharmaceutical composition comprising from about 1 mg to about 8000 mg of an active agent, and said kit further comprising from about one to about eighty-four additional dose of an active agent given on an every other day schedule.

35. The kit of claim 34, further comprising from about 7 to about 100 daily doses of a probiotic.

36. The kit of claim 35, wherein said gastrointestinal disorder is selected from the group consisting of inflammatory bowel disease, Crohn's disease, ulcerative colitis, inflammatory bowel disorder, irritable bowel syndrome, irritable bowel syndrome-diarrhea, irritable bowel syndrome-constipation, irritable bowel syndrome-mixed, irritable bowel syndrome-alternating, dyspepsia, gastro-esophageal reflux, diverticulitis, diverticular disease, gastroparesis, microscopic colitis, lymphocytic colitis, collagenous colitis, indeterminant colitis, eosinophilic esophagitis, HIV-associated diarrhea, antibiotic-associated colitis, clostridium difficile-associated diarrhea, pseudo-membranous colitis, diarrhea associated with immunodeficiency disorders, small bowel overgrowth syndrome, celiac disease, Whipple's disease, CMV-associated colitis, Behcet's syndrome, and combinations thereof.

37. A pharmaceutical composition comprising; from about 1 mg to about 8000 mg of an salicyclic derivative, at least about 10³ cfu of a probiotic; wherein said probiotic is selected from the group consisting of Lactobacillus spp., Bifidobacterium spp., or combinations thereof; and wherein said composition is orally administered on less than daily basis.
